(19) United States
(12) Patent Application Publication
(10) Pub. No.: US 2024/0350443 A1
(43) Pub. Date: Oct. 24, 2024

(54) COMPOSITION, FORMULATION, PREPARATION METHOD AND USE FOR GLUTATHIONE PRECURSOR

(71) Applicant: WUHAN GRAND HOYO CO., LTD., Hubei (CN)

(72) Inventors: Lei YANG, Hubei (CN); Chengjun ZHU, Hubei (CN); Wenwen ZHANG, Hubei (CN); Panpan XING, Hubei (CN); Xiaomeng WANG, Hubei (CN); Haixia SU, Hubei (CN); Liyan ZHA, Hubei (CN); Dawei LIU, Hubei (CN); Hao FENG, Hubei (CN)

(21) Appl. No.: 18/294,559

(22) PCT Filed: Aug. 9, 2022
(86) PCT No.: PCT/CN2022/111265

§ 371 Date: Feb. 2, 2024

(30) Foreign Application Priority Data
Aug. 10, 2021 (CN) 202110915843.4

(51) Int. Cl. A61K 31/198; A61K 9/20; A61K 9/48; A61K 31/145; A61K 33/04; A61K 33/30; A61P 1/16

(52) U.S. Cl. CPC A61K 31/198; A61K 9/20; A61K 9/2013; A61K 9/2027; A61K 9/2054; A61K 9/2059; A61K 9/2068; A61K 9/4833; A61K 9/4858; A61K 9/4866; A61K 31/145; A61K 33/04; A61K 33/30; A61P 1/16

(57) ABSTRACT

A composition, formulation, preparation method and use for a glutathione precursor. The composition comprises, in parts by weight: a selenium-containing compound; a glutathione precursor; a zinc-containing compound. A powder obtained by mixing the present composition has good mixing uniformity and good fluidity, and facilitates filling in a filling capsule; and a capsule containing the composition has the effects of liver protection, oxidation resistance, and immunity enhancement.

The present application claims the priority of Chinese Patent Application No. 2021109158434 filed on Aug. 10, 2021, the content of which is incorporated herein by reference in its entirety.

### TECHNICAL FIELD

The present disclosure relates to a composition of a glutathione precursor, and a formulation thereof, a preparation method therefor and use thereof.

### BACKGROUND

With increasingly fierce competition and industrial waste gas emission in modern society, more and more people have various sub-health manifestations of fatigue and weakness, liver function decline, poor sleep, emotional instability, hypomnesia and the like. The causes and mechanisms of sub-health states, and the efficacy research of prevention and control measures are all closely related to the action mechanism of free radicals, biological anti-oxidation intervention, the anti-oxidation effect and immunity.

Among them, the liver mainly functions as a participant in substance metabolism, biotransformation (detoxification and inactivation), generation and removal of blood coagulation substances, and generation and discharge of bile. The liver is rich in mononuclear phagocytes and plays an important role in specific and non-specific immunity.

A series of researches on immunity enhancement, oxidation resistance, and liver-protecting are reported in the prior art:

WO2020174339A1 disclosed an antioxidant composition for increasing vitamin levels and reducing oxidative damage in a subject, the composition comprising an active agent comprising polydatin and acetylcysteine.

CN1829453A disclosed a composition for treating or preventing infection and enhancing immunity, which comprises a selenium compound (e.g., selenium yeast complex), a glutathione precursor (e.g., acetylcysteine), an alkalinity-enhancing component, a sulfur source, and the like.

CN103479833B disclosed a composition for protecting a liver, which is prepared from the following raw materials: a milk thistle extract, a green tea extract, an acai berry extract, an α-lipoic acid, and N-acetyl-L-cysteine. Through combined use of the milk thistle extract, the green tea extract, the acai berry extract, the α-lipoic acid and the N-acetyl-L-cysteine, the present disclosure has a significant liver protection effect, and can be used for treating and/or preventing acute liver damage and chronic liver damage.

CN100584327C disclosed a pharmaceutical composition for treating liver diseases, which comprises a composition of acetylcysteine or a pharmaceutically acceptable salt thereof and at least one drug for treating liver diseases, wherein the drug for treating liver diseases is selected from: oxymatrine, tiopronin, or monoammonium glycyrrhizinate. The pharmaceutical composition has a good liver protection effect, and can be used for early treatment of liver failure on the basis of comprehensive treatment.

It can be known from the above prior art that the existing drug has a single effect, can only be used for enhancing immunity, or treating or protecting the liver, and thus is difficult to meet the comprehensive requirements of sub-health groups, namely, multiple requirements of protecting the liver, enhancing immunity, resisting oxidation and the like.

### SUMMARY OF THE INVENTION

The technical problem to be solved in the present disclosure is for overcoming the fact that the drug in the prior art has a single effect and cannot meet the comprehensive requirements of sub-health groups, and thus the present disclosure provides a composition of a glutathione precursor, and a formulation thereof, a preparation method therefor and use thereof. The formulation prepared from the composition of a glutathione precursor can achieve multiple effects of liver protection, oxidation resistance and immune efficacy.

The present disclosure solves the above problems by the following technical solutions:

The present disclosure provides a composition of a glutathione precursor, which comprises the following components: a selenium-containing compound, a glutathione precursor, and a zinc-containing compound.

In the present disclosure, preferably, the composition of a glutathione precursor comprises the following components in parts by weight: 5-50 parts of the selenium-containing compound, 200-1200 parts of the glutathione precursor, and 20-105 parts of the zinc-containing compound.

In the present disclosure, the selenium-containing compound refers to a selenium-containing component, and is not limited thereto by the conventional definition of the compound.

In the present disclosure, the selenium-containing compound preferably comprises one or more of sodium selenite, L-selenium-methylselenocysteine and a selenium-enriched yeast, and is more preferably a selenium-enriched yeast.

In the present disclosure, the amount of the selenium-containing compound is preferably 20-50 parts by weight, more preferably 30-50 parts by weight, for example, 45 parts by weight.

In the present disclosure, the particle size of the selenium-containing compound is preferably 40-120 meshes, and more preferably 60-80 meshes.

In the present disclosure, the glutathione precursor preferably comprises cysteine and/or N-acetylcysteine, and is more preferably N-acetylcysteine.

In the present disclosure, the amount of the glutathione precursor is preferably 400-1200 parts by weight, for example, 450 parts, 495 parts, 515 parts, 525 parts, 650 parts or 700 parts by weight, and more preferably 600-800 parts by weight.

In the present disclosure, the particle size of the glutathione precursor is preferably 10-140 meshes, more preferably 20-80 meshes, and for example, 40 meshes or 60 meshes.

In the present disclosure, the zinc-containing compound preferably comprises one or more of zinc gluconate, zinc sulfate, zinc citrate, zinc oxide and zinc lactate, and is more preferably zinc gluconate.

In the present disclosure, the amount of the zinc-containing compound is preferably 30-105 parts by weight, for example, 50 parts by weight, and more preferably 70-105 parts by weight.

In the present disclosure, the particle size of the zinc-containing compound is preferably 40-120 meshes, and more preferably 60-80 meshes.

In the present disclosure, preferably, the composition of a glutathione precursor comprises 500-700 parts of N-acetylcysteine or cysteine, 5-30 parts of a selenium-enriched yeast or L-selenium-methylselenocysteine, and 20-70 parts of zinc lactate or zinc gluconate.

In a preferred embodiment of the present disclosure, the composition of a glutathione precursor comprises a selenium-enriched yeast, N-acetylcysteine and zinc gluconate.

In a more preferred embodiment of the present disclosure, the composition of a glutathione precursor consists of a selenium-enriched yeast, N-acetylcysteine and zinc gluconate.

In a more preferred embodiment of the present disclosure, the composition of a glutathione precursor comprises 25-35 parts of a selenium-enriched yeast, 500-700 parts of N-acetylcysteine, and 60-80 parts of zinc gluconate.

In a more preferred embodiment of the present disclosure, the composition of a glutathione precursor comprises 30 parts of a selenium-enriched yeast, 600 parts of N-acetylcysteine, and 70 parts of zinc gluconate.

In a more preferred embodiment of the present disclosure, the composition of a glutathione precursor comprises 45 parts of a selenium-enriched yeast, 600 parts of N-acetylcysteine, and 50 parts of zinc gluconate.

In a preferred embodiment of the present disclosure, the composition of a glutathione precursor comprises a selenium-enriched yeast, N-acetylcysteine and zinc lactate.

In a more preferred embodiment of the present disclosure, the composition of a glutathione precursor comprises 50 parts of a selenium-enriched yeast, 495 parts of N-acetylcysteine, and 105 parts of zinc lactate.

In a preferred embodiment of the present disclosure, the composition of a glutathione precursor comprises sodium selenite, cysteine and zinc oxide.

In a more preferred embodiment of the present disclosure, the composition of a glutathione precursor comprises 30 parts of sodium selenite, 525 parts of cysteine, and 105 parts of zinc oxide.

In a preferred embodiment of the present disclosure, the composition of a glutathione precursor comprises sodium selenite, cysteine and zinc sulfate.

In a more preferred embodiment of the present disclosure, the composition of a glutathione precursor comprises 50 parts of sodium selenite, 515 parts of cysteine, and 105 parts of zinc sulfate.

In a more preferred embodiment of the present disclosure, the composition of a glutathione precursor comprises 50 parts of sodium selenite, 450 parts of acetylcysteine, and 105 parts of zinc sulfate.

In a preferred embodiment of the present disclosure, the composition of a glutathione precursor comprises L-selenium-methylselenocysteine, cysteine and zinc gluconate.

In a more preferred embodiment of the present disclosure, the composition of a glutathione precursor comprises 5 parts of L-selenium-methylselenocysteine, 650 parts of cysteine, and 50 parts of zinc gluconate.

In a preferred embodiment of the present disclosure, the composition of a glutathione precursor comprises L-selenium-methylselenocysteine, N-acetylcysteine and zinc lactate.

In a more preferred embodiment of the present disclosure, the composition of a glutathione precursor comprises 5 parts of L-selenium-methylselenocysteine, 700 parts of N-acetylcysteine, and 20 parts of zinc lactate.

In the present disclosure, the composition of a glutathione precursor preferably further comprises a component A comprising one or more of vitamins, taurine, fatty acids, proteins, probiotics, polypeptides, lipoic acid, coenzyme Q10, arginine, ornithine, citrulline, glutamine, isoleucine, valine, leucine, and histidine.

Where preferably, the content of the component A is 1-60 parts by weight, for example, 40 parts by weight.

In the present disclosure, preferably, the composition of a glutathione precursor further comprises a flavoring agent.

Where preferably, the content of the flavoring agent is 10-30 parts by weight, for example, 15 parts or 20 parts by weight.

Where the particle size of the flavoring agent is preferably 40-120 meshes, and more preferably 40-60 meshes.

Where preferably, the flavoring agent comprises one or more of a green tea essence, a sweet orange essence, and a blueberry essence.

When the flavoring agent is a green tea essence, the content of the green tea essence is preferably 10-20 parts by weight, for example, 15 parts by weight.

When the flavoring agent is a sweet orange essence, the content of the sweet orange essence is preferably 10-30 parts by weight, for example, 20 parts by weight.

When the flavoring agent is a blueberry essence, the content of the blueberry essence is preferably 10-30 parts by weight, for example, 20 parts by weight.

In the present disclosure, the weight parts of the component A and the flavoring agent are relative to the weight parts of the components in the composition of a glutathione precursor.

The present disclosure further provides a method for preparing the composition of a glutathione precursor, which comprises the following steps: mixing the above components.

The present disclosure further provides use of the composition of a glutathione precursor as a pharmaceutically active ingredient in the manufacture of a medicament for protecting a liver, resisting oxidation and enhancing immunity.

The present disclosure further provides a pharmaceutical formulation, wherein raw materials thereof comprise the composition of a glutathione precursor described above.

In the present disclosure, the pharmaceutical formulation may be in a dosage form conventional in the art, such as a powder, a granule, a tablet, a capsule formulation, an oral liquid, a dry suspension, or other pharmaceutical formulations, and preferably a capsule formulation.

In the present disclosure, the raw materials of the pharmaceutical formulation may further comprise an additive at an addition amount which is conventional in the art, such as one or more of a diluent, a binder, a disintegrant, a lubricant, a glidant, and a filler. The diluent may be sodium carboxymethyl starch. The binder may be povidone. The filler may be microcrystalline cellulose. The lubricant may be magnesium stearate. The glidant may be silicon dioxide and/or talcum powder. Talcum powder, when eaten in excess or for a long period of time, may easily cause gum bleeding or canker sore, so the preferred glidant is silicon dioxide.

Where when the pharmaceutical formulation comprises the lubricant, the content of the lubricant is preferably 3-8 parts by weight, for example, 3.75 parts, 4.5 parts, 5.5 parts, 5.63 parts, 6.5 parts, or 7.5 parts by weight.

Where when the pharmaceutical formulation comprises the glidant, the content of the glidant is preferably 10-20 parts by weight, for example, 15 parts by weight.

Where when the pharmaceutical formulation comprises the filler, preferably, the content of the filler is 1-100 parts by weight, for example, 1.25-37.5 parts or 2.5-22.5 parts by weight, and for another example, 7.5 parts, 8.5 parts, 9.37 parts, 12.5 parts, 14.5 parts, 17.5 parts, 22 parts, 27.5 parts, or 30.5 parts by weight. The particle size of the filler is preferably 40-120 meshes, and more preferably 60-80 meshes.

Where when the pharmaceutical formulation comprises the diluent, the content of the diluent is preferably 1-40 parts by weight, for example, 5 parts by weight.

Where when the pharmaceutical formulation comprises the binder, the content of the binder is preferably 1-30 parts by weight, for example, 5 parts by weight.

In a preferred embodiment of the present disclosure, the pharmaceutical formulation is a capsule formulation, and raw materials of the capsule formulation further comprise a lubricant, a glidant, a flavoring agent, and a filler.

Where the content of the lubricant is preferably 3-8 parts by weight, for example, 3.75 parts, 5.63 parts, or 7.5 parts by weight.

Where the content of the glidant is preferably 10-20 parts by weight, for example, 15 parts by weight.

Where the content of the filler is preferably 1-100 parts by weight, for example, 1.25-37.5 parts or 2.5-22.5 parts by weight, and for another example, 7.5 parts, 12.5 parts, 22 parts or 17.5 parts by weight. The particle size of the filler is preferably 40-120 meshes, and more preferably 60-80 meshes.

Where the volume of the capsule shell in the capsule formulation is determined according to the net weight of the finally prepared capsule formulation, for example, the volume of the capsule shell in the capsule with the net weight of 0.75 g/capsule is 0.95 mL.

Where preferably, the capsule formulation is prepared by the following preparation method: filling a mixed powder of the glutathione precursor, the zinc-containing compound, the selenium-containing compound, the flavoring agent, the lubricant, the glidant and the filler into a capsule.

More preferably, the capsule formulation is prepared by the following preparation method: filling a mixed powder of the N-acetylcysteine, the zinc gluconate, the selenium-enriched yeast, the green tea essence, the magnesium stearate, the silicon dioxide and the microcrystalline cellulose into a capsule.

Where preferably, the capsule formulation is prepared by the following preparation method: mixing a first mixed powder of the zinc-containing compound, the selenium-containing compound, the flavoring agent and the filler with a mixed powder of the glutathione precursor to obtain a second mixed powder, then mixing the second mixed powder with the lubricant and the glidant to obtain a total mixed powder, and filling the total mixed powder into a capsule.

Before the first mixed powder is obtained, preferably, the zinc-containing compound and the selenium-containing compound are sieved through a 60-80-mesh sieve. For the zinc-containing compound and the selenium-containing compound, when the zinc gluconate and the selenium-enriched yeast are sieved through a 60-mesh sieve, the sieving rate is relatively high and is 98% or above, and they may pass through the sieve easily, with the sieving time being short; when they are sieved through an 80-mesh sieve, the sieving rate is relatively low and is 96% or above, and they may pass through the sieve easily, with the sieving time being long. Before the first mixed powder is obtained, preferably, the filler is sieved through a 60-80-mesh sieve. Before the first mixed powder is obtained, preferably, the flavoring agent is sieved through a 40-60-mesh sieve.

Before the second mixed powder is obtained, the glutathione precursor is preferably sieved through a 20-80-mesh sieve, and is more preferably sieved through a 40-80-mesh sieve.

More preferably, the capsule formulation is prepared by the following preparation method: mixing a first mixed powder of the zinc gluconate, the selenium-enriched yeast, the green tea essence and the microcrystalline cellulose with a mixed powder of the N-acetylcysteine to obtain a second mixed powder, then mixing the second mixed powder with the magnesium stearate and the silicon dioxide to obtain a total mixed powder, and filling the total mixed powder into a capsule.

In a preferred embodiment of the present disclosure, the pharmaceutical formulation is a tablet, and raw materials of the tablet further comprise a lubricant, a glidant, a flavoring agent, and a filler.

Where the content of the lubricant is preferably 3-8 parts by weight, for example, 3.75 parts, 4.5 parts, 5.5 parts, 5.63 parts, 6.5 parts, or 7.5 parts by weight.

Where the content of the glidant is preferably 10-20 parts by weight, for example, 15 parts by weight.

Where the content of the filler is preferably 1-100 parts by weight, for example, 2.5-30.5 parts by weight, and for another example, 8.5 parts, 9.37 parts or 14.5 parts by weight. The particle size of the filler is preferably 40-120 meshes, and more preferably 60-80 meshes.

Where preferably, the tablet is prepared by the following preparation method: tabletting a mixed powder of the glutathione precursor, the zinc-containing compound, the selenium-containing compound, the flavoring agent, the lubricant, the glidant and the filler.

More preferably, the tablet is prepared by the following preparation method: mixing a first mixed powder of the zinc-containing compound, the selenium-containing compound, the flavoring agent and the filler with a mixed powder of the glutathione precursor to obtain a second mixed powder, then mixing the second mixed powder with the lubricant and the glidant to obtain a total mixed powder, and tabletting the total mixed powder.

Before the first mixed powder is obtained, preferably, the zinc-containing compound and the selenium-containing compound are sieved through a 60-80-mesh sieve.

Before the first mixed powder is obtained, preferably, the filler is sieved through a 60-80-mesh sieve.

Before the first mixed powder is obtained, preferably, the flavoring agent is sieved through a 40-60-mesh sieve.

Before the second mixed powder is obtained, the glutathione precursor is preferably sieved through a 20-80-mesh sieve, and is more preferably sieved through a 40-80-mesh sieve.

Where the tablet may have a net weight conventional in the art, for example, a net weight of 0.75 g/tablet.

Where the tablet may have the hardness conventional in the art, for example, the hardness of 100-160 N.

The tabletting step may be carried out according to the net weight and the hardness of the tablet.

In the present disclosure, “parts by weight” reflects the relationship between the amounts of the components, and the specific amounts of the components are not limited thereto, and those skilled in the art may appropriately adjust the specific amounts according to the relationship between the amounts.

The present disclosure further provides use of the composition of a glutathione precursor described above or the pharmaceutical formulation described above in liver protection, oxidation resistance or immunity enhancement.

The present disclosure further provides a method for protecting a liver, resisting oxidation or enhancing immunity, which comprises administering to a patient a medicament comprising the composition of a glutathione precursor described above or the pharmaceutical formulation described above.

On the basis of the general knowledge in the art, the above preferred conditions can be combined arbitrarily to obtain preferred embodiments of the present disclosure.

The reagents and raw materials used in the present disclosure are commercially available.

The positive and progressive effects of the present disclosure are as follows:
(1) The combined use of the glutathione precursor, the zinc-containing compound and the selenium-containing compound achieves the effects of liver protection, oxidation resistance or immunity enhancement, and especially the cooperative effect of the N-acetylcysteine, the zinc gluconate and the selenium-enriched yeast is more excellent;
(2) In the preferred embodiments of the present disclosure, the green tea essence is added as a flavoring agent, which can cover the unpleasant odor of the raw materials of the glutathione precursor and has the fragrance of green tea, such that a user can swallow the drug easily and the taking feeling is good;
(3) In the preferred embodiments of the present disclosure, the capsule formulation has a simple preparation process, and after the capsule formulation is taken, the content is easy to disperse and dissolve out in the digestive tract, so that the absorption rate is relatively good; the capsule shell protects the drug from oxygen and light in the air, thereby improving the stability of the drug; and
(4) The mixed powder of the composition of a glutathione precursor has good mixing uniformity and good fluidity and is conveniently filled into a capsule.

### BRIEF DESCRIPTION OF THE DRAWING

FIG. 1 is the histopathological examination pictures of each group of compositions of a glutathione precursor in the liver protection function experiment in Example 28, wherein, A-I correspond to A: livers in the blank control group (oil red O staining×200), B: livers in the model control group (oil red O staining×200), C: livers in the experiment group 1 (oil red O staining×200), D: livers in the experiment group 2 (oil red O staining×200), E: livers in the experiment group 3 (oil red O staining×200), F: livers in the experiment group 4 (oil red O staining×200), G: livers in the experiment group 5 (oil red O staining×200), H: livers in the experiment group 6 (oil red O staining×200), and I: livers in the experiment group 7 (oil red O staining×200).

### DETAILED DESCRIPTION

The present disclosure is further illustrated by the following embodiments, which are not intended to limit the present disclosure. Experimental procedures without specified conditions in the following examples were conducted in accordance with conventional procedures and conditions, or in accordance with the manufacturer's manual.

The sample numbers in the following examples are for convenience of description only, and are not intended to limit the specific samples.

The angle of repose was measured as follows for the following examples:

An iron ring was fixed on an iron support, a funnel was placed in the iron ring, a culture dish was put upside down directly below the funnel, and the culture dish was adjusted to make the central dot thereof perpendicular to the funnel; materials were slowly added into the funnel until the materials flowed down at the edge of the culture dish, then feeding was stopped, the stacking height H of the materials was measured with a ruler, and then the diameter R of the culture dish was measured.

The calculation formula was as follows: tan α=2H÷Rα=Arctan α;

The smaller the angle of repose of the materials was, the smaller the friction force was, and the better the fluidity was; the fluidity was the best when the angle was less than 30°, and the materials could be used for industrial production when the angle was less than 40°.

The selenium-enriched yeast was available from Angel Yeast Co., Ltd., and the zinc gluconate was available from Zhengzhou Ruipu Biological Engineering Co., Ltd.

### Examples 1-4: Preparation of Composition of N-acetylcysteine

Raw materials and dosage were as follows: 600 g of N-acetylcysteine, 70 g of zinc gluconate, and 30 g of a selenium-enriched yeast.

The preparation process was as follows: except that the N-acetylcysteine was not pulverized and directly sieved in Example 1, the following components were respectively pulverized and sieved according to Table 1 in other examples to obtain powders; and then the powders were weighed according to the above dosage, and mixed to obtain the composition of N-acetylcysteine.

Three samples of each of Examples 1-4 were tested for angle of repose and relative standard deviation (RSD) of the composition, and the results are shown in Table 1 below.

TABLE 1

| | | Sieving mesh | | Angle of repose | | | | |
|---|---|---|---|---|---|---|---|---|
| | Sieving mesh | number of | Sieving mesh | of the composition/° | | | | |
| | number of zinc | selenium- | number of N- | Sample | Sample | Sample | Mean | |
| No. | gluconate | enriched yeast | acetylcysteine | 1 | 2 | 3 | value/° | RSD/% |
| Example 1 | 60 meshes | 60 meshes | Not pulverized | 41.1 | 44.1 | 43.1 | 42.8 | 3.5 |
| | | | (20 meshes) | | | | | |
| Example 2 | 60 meshes | 60 meshes | 40 meshes | 45.9 | 43.5 | 44.7 | 44.7 | 2.7 |
| Example 3 | 60 meshes | 60 meshes | 60 meshes | 47.9 | 49.9 | 49.4 | 49.1 | 2.2 |
| Example 4 | 60 meshes | 60 meshes | 80 meshes | 54.1 | 52.8 | 54.7 | 53.8 | 1.8 |

As can be seen from the experimental results in Table 1, the angle of repose of the mixed powder gradually increased with the decrease in the pulverized particle size, indicating that the fluidity of the mixed powder gradually deteriorated; when the N-acetylcysteine was not pulverized (20 meshes), the fluidity of the mixed powder was the best, but as the pretreatment of the zinc gluconate and the selenium-enriched yeast was carried out by sieving through a 60-mesh sieve, the particle size difference was relatively large, and if the N-acetylcysteine was not pulverized, the conditions of sample layering and uneven content were easy to occur in the mechanical capsule filling process.

### Examples 5-7: Preparation of Capsule Formulation

Raw materials and dosage were as follows: 600 g of N-acetylcysteine, 70 g of zinc gluconate, 30 g of a selenium-enriched yeast, 15 g of a green tea essence, 17.5 g of microcrystalline cellulose, 10 g of silicon dioxide, and 7.5 g of magnesium stearate.

The preparation method was as follows:
(1) the above components were respectively pulverized, the N-acetylcysteine and the green tea essence were sieved through a 40-mesh sieve, and the zinc gluconate, the selenium-enriched yeast and the microcrystalline cellulose were respectively sieved through a 60-mesh sieve to obtain powders for later use;
(2) the powders prepared in the step (1) were weighed according to the above dosage;
(3) the zinc gluconate powder, the selenium-enriched yeast powder, the green tea essence powder and the microcrystalline cellulose powder were mixed for 10 min to obtain a first mixed powder; then the first mixed powder and the N-acetylcysteine powder were mixed for t min according to the time in Table 2 to obtain a second mixed powder; and the second mixed powder and the silicon dioxide and the magnesium stearate were mixed for 10 min to obtain a total mixed powder;
(4) capsule filling: the total mixed powder was filled into a capsule according to the net weight of 0.75 g/capsule, wherein the volume of the hollow capsule shell was 0.95 mL (see below for capsule shell selection); and
(5) packaging: the powder on the surface of the capsule was removed, and rejected products were picked off and bottled in polyethylene bottles.

TABLE 2

| | | | Selenium | | |
|---|---|---|---|---|---|
| | Mixing | Sample | content | Mean value | |
| No. | time t | No. | (mg/100 g) | (mg/100 g) | RSD/% |
| Example 5 | 10 min | 5-1 | 7.92 | 8.27 | 4.23 |
| | | 5-2 | 8.94 | | |
| | | 5-3 | 8.23 | | |
| | | 5-4 | 8.12 | | |
| | | 5-5 | 8.16 | | |
| | | 5-6 | 8.24 | | |
| Example 6 | 20 min | 6-1 | 8.18 | 8.06 | 1.71 |
| | | 6-2 | 8.16 | | |
| | | 6-3 | 7.85 | | |
| | | 6-4 | 8.03 | | |
| | | 6-5 | 7.98 | | |
| | | 6-6 | 8.18 | | |
| Example 7 | 30 min | 7-1 | 8.10 | 8.28 | 2.47 |
| | | 7-2 | 8.19 | | |
| | | 7-3 | 8.64 | | |
| | | 7-4 | 8.31 | | |
| | | 7-5 | 8.37 | | |
| | | 7-6 | 8.10 | | |

Six samples of each of Examples 5-7 were tested for the selenium content in each sample, the RSD value for each example was calculated, and the specific results are shown in Table 2.

As can be seen from Table 2, when the materials were mixed for 10 min, the RSD value for the selenium content at 6 sampling points was 4.23%, which was less than 5%, indicating that the materials were mixed well when mixed for 10 min. If mass production was required, in order to ensure that the materials could be uniformly mixed during mass production, the mixing time could be set to be 20 min.

### Where the Selection Process of the Capsule Shell of the Capsule Formulation in the Step (3) was as Follows:

Taking Example 6 as an example, the mixed powder obtained in Example 6 was tested for bulk density and tap density.

Bulk density test was as follows: a clean and dry 100 mL measuring cylinder was taken and placed on a balance, and the reading of the balance was reset to zero; the mixed powder prepared in Example 6 was gently and slowly moved to the measuring cylinder to a scale of 50 mL, the volume was read, and the weights of the samples were weighed, and the measurement was repeated for three times (samples 1-3), the density was calculated, and the mean value was calculated; the results are shown in Table 3;

Tap density measurement method was as follows: the measuring cylinder containing the samples was placed on a table (with a cloth of about 5 mm thickness laid), and the measuring cylinder was dropped onto the table from a height of about 2 cm, and this operation was repeated for about 100 times, the volume was read, and the density was calculated; the results are shown in Table 3.

TABLE 3

| | | Direct loading | | After the materials | |
|---|---|---|---|---|---|
| | | of materials | | were compacted | |
| Serial | Weight | Volume | Bulk density | Volume | Tap density |
| number | (g) | (mL) | (g/mL) | (mL) | (g/mL) |
| Sample 1 | 37.62 | 50.0 | 0.752 | 36.5 | 1.031 |
| Sample 2 | 35.09 | 47.0 | 0.747 | 33.5 | 1.047 |
| Sample 3 | 36.41 | 49.5 | 0.736 | 35.5 | 1.026 |

| | | |
|---|---|---|
| Average density (g/mL) | 0.745 | 1.035 |

According to the formula of Example 6, the specification of the capsule formulation was 0.75 g/capsule, and meanwhile, the required volume range of the hollow capsule was calculated to be 0.725-1.007 mL according to the measured bulk density and tap density; according to data query, the volume of the 0 #hollow capsule shell was 0.68 mL, the volume of the 00 #hollow capsule shell was 0.95 mL, and therefore the 00 #hollow capsule was selected for filling.

### Examples 8-9: Preparation of Capsule Formulation

600 g of N-acetylcysteine, 70 g of zinc gluconate, 30 g of a selenium-enriched yeast, 15 g of a green tea essence, 7.5 g of magnesium stearate, and 7.5-17.5 g of microcrystalline cellulose (the total weight of the microcrystalline cellulose was supplemented to 750 g), the amounts of silicon dioxide and microcrystalline cellulose are shown in the table below, and other preparation processes are consistent with those in Example 6; three samples were taken for each example, and the measured angle of repose and the RSD value of the total mixed powder are shown in Table 4 below.

TABLE 4

| | Silicon | Microcrystalline | | | | | |
|---|---|---|---|---|---|---|---|
| | dioxide amount | cellulose | Angle of repose/° | | | Mean | |
| No. | (g) | (g) | Sample 1 | Sample 2 | Sample 3 | value/° | RSD/% |
| Example 6 | 10 | 17.5 | 39.7 | 39.4 | 38.7 | 39.3 | 1.4 |
| Example 8 | 15 | 12.5 | 39.4 | 39.7 | 37.9 | 39.0 | 2.5 |
| Example 9 | 20 | 7.5 | 37.5 | 39.0 | 39.7 | 38.8 | 2.9 |

As can be seen from the experimental results of Table 4, there was no significant difference in the angle of repose of the total mixed powder.

### Examples 10-11: Preparation of Capsule Formulation

A capsule filling pre-experiment was carried out by using a full-automatic capsule machine, the machine was stopped running to take down the filling rod for observation in the case that the equipment ran for a period of time and was at a standstill, and it was found that a large amount of materials were adhered to the filling rod and were difficult to clean; the adhesion of the materials to the filling rod caused an increase in the friction between the filling rod and the hole walls of the die, such that the operation of the equipment was at a standstill. In order to reduce the adhesion of the materials to the filling rod and reduce the friction between the materials and the hole walls of the die during the capsule filling process, so as to ensure that the filling of the capsule was smoothly performed, a lubricant magnesium stearate was added to the raw materials, wherein the addition amount of the magnesium stearate is shown in Table 5 (microcrystalline cellulose was supplemented until the total weight was 750 g), other conditions were consistent with those of the preparation process in Example 6, and the experimental results are shown in Table 5.

TABLE 5

| | Magnesium | |
|---|---|---|
| | stearate | Material adhesion between the filling rod and |
| No. | amount (g) | the hole walls of the die |
| Example 10 | 3.75 | Obvious material adhesion between the |
| | | filling rod and the hole walls of the die |
| Example 11 | 5.63 | Adhesion of an extremely small amount of |
| | | materials between the filling rod and the |
| | | hole walls of the die |
| Example 6 | 7.5 | No obvious material adhesion between the |
| | | filling rod and the hole walls of the die |

### Examples 12-19: Preparation of Capsule Formulation

As the raw materials of the N-acetylcysteine has a strong garlic-like odor which may cause cough, nausea, vomit, halitosis and the like, and causes a user to easily feel uncomfortable, a proper flavoring agent was added into the raw material mixed powder to cover the unpleasant odor of the raw materials of the N-acetylcysteine; the type and amount of the flavoring agent added are as shown in Table 6 (microcrystalline cellulose was supplemented until the total weight was 750 g), and other conditions were consistent with those of Example 6.

TABLE 6

| | Type of | Amount | |
|---|---|---|---|
| No. | flavoring agent | (g) | Odor |
| Example 12 | Green tea | 10 | No obvious N-acetylcysteine odor, and slight green tea fragrance |
| Example 6 | essence | 15 | No obvious N-acetylcysteine odor, and obvious green tea fragrance |
| Example 13 | | 20 | No obvious N-acetylcysteine odor, and slightly heavy green tea fragrance |
| Example 14 | Sweet orange | 10 | Obvious N-acetylcysteine odor, and no sweet orange essence odor |
| Example 15 | essence | 20 | Obvious N-acetylcysteine odor, and slight sweet orange essence odor |
| Example 16 | | 30 | No obvious N-acetylcysteine odor, and slight sweet orange fragrance |
| Example 17 | Blueberry | 10 | No obvious blueberry fragrance, and obvious N-acetylcysteine odor |
| Example 18 | essence | 20 | No obvious blueberry fragrance, and obvious N-acetylcysteine odor |
| Example 19 | | 30 | Slight blueberry fragrance, and slightly obvious N-acetylcysteine odor |

As can be seen from the experimental results in Table 6, the odor covering effect of the green tea essence was generally better than that of the sweet orange essence and that of the blueberry essence, the sweet orange essence had a significant odor covering effect only when the dose was 30 g, while the green tea essence could completely cover the acetylcysteine odor when the dose was 15 g.

### Example 20 (Tablet)

Provided is a composition of a glutathione precursor with liver protection, oxidation resistance and immunity enhancement functions, which comprises the following components: 525 g of cysteine, 105 g of zinc oxide, 30 g of sodium selenite, 30 g of a sweet orange essence, 4.5 g of magnesium stearate, 30.5 g of microcrystalline cellulose, 20 g of silicon dioxide, and 5 g of sodium carboxymethyl starch.

The method for preparing a tablet was as follows:
(1) The cysteine and the flavoring agent were sieved through a 40-mesh sieve, and the zinc oxide, the sodium selenite and the microcrystalline cellulose were respectively sieved through a 60-mesh sieve to obtain powders for later use;
(2) The powders prepared in the step (1) were weighed according to the weight of the above components;
(3) Firstly, the zinc oxide powder, the sodium selenite powder, the flavoring agent powder and the microcrystalline cellulose powder were mixed for 10 min to obtain a first mixed powder; then the first mixed powder and the cysteine powder were mixed for 10 min to obtain a second mixed powder; and the second mixed powder and the silicon dioxide and the magnesium stearate were mixed for 10 min to obtain a total mixed powder; and
(4) Tabletting: tabletting was carried out according to the net weight of 0.75 g/tablet, and the hardness of the tablet was controlled to be 100-160 N.

### Example 21 (Tablet)

Provided is a composition of a glutathione precursor with liver protection, oxidation resistance and immunity enhancement functions, which comprises the following components: 650 g of cysteine, 50 g of zinc gluconate, 5 g of L-selenium-methylselenocysteine, 10 g of a blueberry essence, 6.5 g of magnesium stearate, 8.5 g of microcrystalline cellulose, 15 g of silicon dioxide, and 5 g of povidone.

The method for preparing a tablet was the same as that of Example 20.

### Example 22 (Tablet)

Provided is a composition of a glutathione precursor with liver protection, oxidation resistance and immunity enhancement functions, which comprises the following components: 600 of N-acetylcysteine, 50 of zinc gluconate, 45 of a selenium-enriched yeast, 20 of a green tea essence, 5.5 of magnesium stearate, 14.5 of microcrystalline cellulose, and 15 of silicon dioxide.

The method for preparing a tablet was the same as that of Example 20.

### Example 23 (Tablet)

Provided is a composition of a glutathione precursor with liver protection, oxidation resistance and immunity enhancement functions, which comprises the following components: 495 g of N-acetylcysteine, 105 g of zinc lactate, 50 g of a selenium-enriched yeast, 20 g of a green tea essence, 5.63 g of stearic acid, 9.37 g of microcrystalline cellulose, 20 g of silicon dioxide, 40 g of taurine, and 5 g of sodium carboxymethyl starch.

The method for preparing a tablet was the same as that of Example 20.

### Example 24 (Capsule)

Provided is a composition of a glutathione precursor with liver protection, oxidation resistance and immunity enhancement functions, which comprises the following components: 700 g of N-acetylcysteine, 20 g of zinc lactate, 5 g of L-selenium-methylselenocysteine, 10 g of a sweet orange essence, 3.75 g of magnesium stearate, 1.25 g of microcrystalline cellulose, and 10 g of silicon dioxide.

The method for preparing a capsule was the same as that of Example 5.

### Example 25 (Capsule)

Provided is a composition of a glutathione precursor with liver protection, oxidation resistance and immunity enhancement functions, which comprises the following components: 515 g of cysteine, 105 g of zinc sulfate, 50 g of sodium selenite, 30 g of a blueberry essence, 8 g of magnesium stearate, 22 g of microcrystalline cellulose, and 20 g of silicon dioxide.

The method for preparing a capsule was the same as that of Example 5.

### Example 26 (Capsule)

Provided is a composition of a glutathione precursor with liver protection, oxidation resistance and immunity enhancement functions, which comprises the following components: 450 g of N-acetylcysteine, 105 g of zinc sulfate, 50 g of sodium selenite, 20 g of a green tea essence, 7.5 g of magnesium stearate, 37.5 g of microcrystalline cellulose, 20 g of silicon dioxide, and 60 g of ornithine.

The method for preparing a capsule was the same as that of Example 5.

### Example 27

The following tests were to test the performance of the total mixed powder and the capsule formulation prepared in Example 6 respectively.

### (1) Testing of Relevant Properties of Total Mixed Powder

### 1. Uniformity:

Two samples were respectively taken from the upper, middle and lower positions of a hopper where the total mixed powder was located, and six samples were taken totally; the selenium content in the samples was detected, the RSD values of the content of the six samples were calculated, and whether the total mixed powder of the materials was uniformly mixed or not was evaluated by taking the RSD values as an index; the experimental results are shown in Table 7.

TABLE 7

| | Selenium | | |
|---|---|---|---|
| | content | Mean value | |
| Sample No. | (mg/100 g) | (mg/100 g) | RSD/% |
| 6-1 | 7.39 | 7.67 | 2.34 |
| 6-2 | 7.58 | | |
| 6-3 | 7.77 | | |
| 6-4 | 7.83 | | |
| 6-5 | 7.60 | | |
| 6-6 | 7.84 | | |

As can be seen from the experimental results, the RSD value of the selenium content of the total mixed powder was 2.34%, which was less than 5%, indicating that the difference of the selenium content of the samples at different sampling points of the total mixed powder in Example 6 was relatively small. Thus, it was found that the total mixed powder was uniformly mixed.

### 2. Measurement of Angle of Repose:

An appropriate amount of three aliquots of samples in the total mixed powder were taken for measuring the angle of repose, and the experimental results are shown in Table 8.

TABLE 8

| Number of | Material | Culture dish | Angle of | Mean | |
|---|---|---|---|---|---|
| measurements | height/cm | diameter/cm | repose/° | value/° | RSD/% |
| 6-1 | 3.65 | 9.50 | 37.5 | 38.2 | 1.5 |
| 6-2 | 3.80 | | 38.7 | | |
| 6-3 | 3.75 | | 38.3 | | |

As can be seen from the experimental results in Table 8, the angle of repose of the total mixed powder was 38.2°, which was less than 40°, indicating that the fluidity of the total mixed powder could meet the requirements of mechanical capsule filling.

### (2) Filling Property Verification for Capsule Formulation

A hollow capsule shell (the volume was 0.95 mL) was filled with the prepared total mixed powder by using a full-automatic capsule filling machine, and after the filling was completed, the filling variation was checked according to the filling variation inspection method of the capsule formulation item of volume IV General Rules of the Pharmacopoeia of the People's Republic of China (2020 edition), wherein the filling variation range was-2.79% to 5.77%, which met the related requirements of the capsule formulation, indicating that the formula and the process were feasible; the verification results are shown in Table 9.

TABLE 9

| | Serial number | | | | | | | | | |
|---|---|---|---|---|---|---|---|---|---|---|
| | Sample | Sample | Sample | Sample | Sample | Sample | Sample | Sample | Sample | Sample |
| | 1 | 2 | 3 | 4 | 5 | 6 | 7 | 8 | 9 | 10 |
| Capsule weight/g | 0.8725 | 0.8578 | 0.9112 | 0.8710 | 0.8668 | 0.8751 | 0.8813 | 0.8488 | 0.8780 | 0.8676 |
| Capsule shell | 0.1204 | 0.1184 | 0.1197 | 0.1201 | 0.1185 | 0.1239 | 0.1206 | 0.1197 | 0.1214 | 0.1184 |
| weight/g | | | | | | | | | | |
| Net weight/g | 0.7521 | 0.7394 | 0.7915 | 0.7509 | 0.7483 | 0.7512 | 0.7607 | 0.7291 | 0.7566 | 0.7492 |
| | Serial number | | | | | | | | | |
| | Sample | Sample | Sample | Sample | Sample | Sample | Sample | Sample | Sample | Sample |
| | 11 | 12 | 13 | 14 | 15 | 16 | 17 | 18 | 19 | 20 |
| Capsule weight/g | 0.8844 | 0.8703 | 0.8738 | 0.8671 | 0.8935 | 0.9136 | 0.8733 | 0.8763 | 0.8861 | 0.8716 |
| Capsule shell | 0.1223 | 0.1207 | 0.1231 | 0.1186 | 0.1199 | 0.1203 | 0.1184 | 0.1202 | 0.1209 | 0.1197 |
| weight/g | | | | | | | | | | |
| Net weight/g | 0.7621 | 0.7496 | 0.7507 | 0.7485 | 0.7736 | 0.7933 | 0.7549 | 0.7561 | 0.7652 | 0.7519 |

### (3) Capsule Formulation Stability Study

The influence factors of the naked sample and the bottled sample were examined by tests. The samples were taken on day 0 and day 10, respectively, and the changes in the external properties of the contents of the samples were observed at high temperature (60° C.), high humidity (90%±5% RH), and strong light (4500 Lx±500 Lx); the results are shown in Table 10.

TABLE 10

| | | | N-acetylcysteine | Moisture | Disintegration |
|---|---|---|---|---|---|
| Sample | Condition | Properties | content (g/100 g) | gain (%) | time limit (min) |
| Day 0 | | The content was a white powder, | 80.02 | — | 13 |
| | | had the special taste and odor of | | | |
| | | the product, and had no peculiar | | | |
| | | odor; no foreign object visible with | | | |
| | | normal vision | | | |
| Naked | High | The capsule shell became brittle | 74.85 | −1.81 | 12 |
| sample | temperature | and the contents slightly | | | |
| | for 10 days | agglomerated | | | |
| | High humidity | The capsule shell had soft texture | 71.25 | 13.28 | 11 |
| | for 10 days | and hygroscopic content | | | |
| | Strong light | No obvious change compared with | 78.96 | 0.18 | 12 |
| | for 10 days | day 0 | | | |
| Bottled | High | No obvious change compared with | 78.98 | −0.17 | 11 |
| sample | temperature | day 0 | | | |
| | for 10 days | | | | |
| | High humidity | No obvious change compared with | 77.77 | 0.19 | 11 |
| | for 10 days | day 0 | | | |
| | Strong light | No obvious change compared with | 80.02 | 0.00 | 12 |
| | for 10 days | day 0 | | | |

Note:
“—” indicates undetectable.

As can be seen from Table 10, the capsule shell of the naked sample became brittle and the content slightly agglomerated under the high temperature condition, and the content of N-acetylcysteine of the sample was more different than that of the sample on day 0; under the high humidity condition, the capsule shell of the naked sample had a soft texture and a hygroscopic content, and the changes in properties were larger than those of day 0; while the properties, the disintegration time limit and the N-acetylcysteine content of the bottled sample were not significantly changed compared with day 0 under the conditions of high temperature and high humidity. Under the condition of strong light irradiation, the properties, the disintegration time limit and the N-acetylcysteine content of the naked sample and the bottled sample were not significantly changed compared with day 0. This suggests that the product was sensitive to high temperature and high humidity in a direct air contact state, and had good stability after being packaged in a polyethylene plastic bottle.

### Example 28

The following experimental groups were subjected to the auxiliary liver protection, oxidation resistance and immune efficacy experiments.

Experimental group 1 (Comparative Example 1): 600 g of N-acetylcysteine.

Experimental group 2 (Comparative Example 2): 600 g of N-acetylcysteine and 70 g of zinc gluconate.

Experimental group 3 (Comparative Example 3): 600 g of N-acetylcysteine and 30 g of a selenium-enriched yeast.

Experimental group 4 (Example 26): 450 g of N-acetylcysteine, 105 g of zinc sulfate, and 50 g of sodium selenite.

Experimental group 5 (Example 24): 700 g of N-acetylcysteine, 5 g of L-selenium-methylselenocysteine, and 20 g of zinc lactate.

Experimental group 6 (Example 5): 600 g of N-acetylcysteine, 30 g of a selenium-enriched yeast, and 70 g of zinc gluconate.

Experimental group 7 (Example 21): 650 g of cysteine, 50 g of zinc gluconate, and 5 g of L-selenium-methylselenocysteine.

The following efficacy experimental data were processed by SPSS software, and by means of ANOVA, Duncan's multiple range test was adopted to perform data analysis on the difference of the effects of different experimental groups at the level of P<0.05. Significant differences between each group of data in the same column were indicated by superscript letters. Differences in the superscript letters among the groups of data in the same column indicated significant differences (p<0.05), and differences among the groups of data in the same column, which contained at least one identical letter, indicated insignificant differences (p>0.05). For example, in the same column, two groups with the superscript letters d and cd respectively had no significant difference, while two groups with the superscript letters d and bc respectively had significant differences; the group with the largest numerical value was marked with the superscript letter a, and the differences between the groups with the superscript letters b, c and d and the group with the superscript letter a sequentially increased relative to the group with the superscript letter a.

### 1. Experiment for the Liver Protection Function of the Composition of a Glutathione Precursor

In the experiment, the auxiliary protection effect of the composition of the present disclosure on alcoholic liver damage was researched by detecting three indicators of liver malondialdehyde (MDA) content, reduced glutathione (GSH) and triglyceride (TG) and histopathologic examination (wherein the lower the MDA and TG contents were, the higher the GSH content was, the better the liver protection effect was). If the three indicators of the liver MDA, the reduced GSH and the TG were positive, or any two of the three indicators of the liver MDA, the reduced GSH and the TG were positive and the histopathological examination result was positive, the composition could be determined to have the auxiliary protection effect on alcoholic liver damage.

Test animals were as follows: male SPF-grade Kunming mice aged 5 to 6 weeks, with body weights of 18-22 g, available from Hubei Provincial Laboratory Animal Public Service Center, Hubei, China [certification No.: SCXK (E) 2020-0018].

The experimental method was as follows: 117 mice were randomly divided into 9 groups, namely a blank control group, a model control group and experimental groups 1-7 (the formula of the test samples was referred to the formula of the experimental groups 1-7), with 13 mice in each group.

Distilled water was given to the blank control group and the model control group, corresponding test substances (the dose of acetylcysteine or cysteine agent in each test substance was 500 mg/kg BW) were given to each experimental group according to the formula, and the intragastric capacity was 20 mL/kg BW. On day 30 of the experiment, the mice of the model control group and each experimental group were intragastrically administered with 14 mL/kg BW of 50% ethanol once, and distilled water with equal volume was given to the blank control group, and then the mice were fasted for 16 hours. The mice were sacrificed by cervical dislocation after weighing.

### 1.1. Contents of MDA, Reduced GSH and TG in Liver Homogenates

0.5 g of liver was taken and added with normal saline to prepare a liver homogenate, and then the MDA, reduced GSH and TG were detected. The detection method and the calculation method for the MDA and reduced GSH were described according to the instruction of a kit from NanJing JianCheng Bioengineering Institute, and the TG was detected by using the Beckman Coulter AU680 Chemistry Analyzer. The results are shown in Table 11.

TABLE 11
Contents of MDA, reduced GSH and TG in liver homogenates
of each group of mice (x̅ ± s)

| | Number | | | |
|---|---|---|---|---|
| | of animals | MDA | Reduced GSH | TG |
| Group | (mice) | (nmol/mgprot) | (mg/gprot) | (mmol/L) |
| Blank control | 13 | 0.73 ± 0.36^{d} | 33.86 ± 5.95^{c} | 1.33 ± 0.61^{cd} |
| Model control | 13 | 1.43 ± 0.36^{a} | 27.87 ± 5.01^{d} | 1.94 ± 0.63^{a} |
| Experimental group 1 | 13 | 0.98 ± 0.28^{bc} | 34.55 ± 9.48^{bc} | 1.52 ± 0.64^{bc} |
| Experimental group 2 | 13 | 0.73 ± 0.18^{d} | 40.73 ± 9.91^{ab} | 1.55 ± 0.38^{abc} |
| Experimental group 3 | 13 | 0.82 ± 0.20^{cd} | 37.93 ± 7.66^{abc} | 1.81 ± 0.76^{ab} |
| Experimental group 4 | 13 | 1.05 ± 0.19^{b} | 37.03 ± 4.32^{abc} | 1.37 ± 0.10^{cd} |
| Experimental group 5 | 13 | 0.89 ± 0.12^{bcd} | 35.36 ± 4.85^{bc} | 1.44 ± 0.13^{bcd} |
| Experimental group 6 | 13 | 0.74 ± 0.32^{d} | 42.05 ± 8.92^{a} | 1.08 ± 0.51^{d} |
| Experimental group 7 | 13 | 1.03 ± 0.16^{bc} | 35.04 ± 8.21^{bc} | 1.47 ± 0.20^{bcd} |

As can be seen from the experimental results, compared with the blank control group, the contents of the MDA and TG in the liver homogenates of the animals in the model control group increased, and the reduced GSH content decreased (P<0.05), indicating that the experimental model was established successfully. Compared with the model control group, the MDA content of each test substance group decreased, and the GSH content increased (P<0.05); the TG content decreased in the experimental groups 1 and 4-7 (P<0.05), and the experimental group 6 and the experimental group 1 had significant differences (P<0.05).

### 1.2. Liver Histopathology

Histopathological observation on materials: a cross-section sample was collected from the middle part of the left lobe of each liver of each group of animals, frozen, sectioned, and stained with Sudan III. The damage degree of hepatocytes was observed under an optical microscope.

Evaluation method and scoring criteria were as follows: microscopic examination was carried out to record pathological changes of the cells from the field of view at one end of the liver, and the whole tissue section was observed continuously with a 40× objective lens. The distribution, extent and area of lipid droplets in the liver were mainly observed.

The areas of the fields of view occupied by various lesions in each field of view were respectively recorded, and the total lesion scores of the observed fields of view were accumulated.

The scoring basis was as follows: the lipid droplets in the hepatocytes were scattered and rare (0 points); there were not more than ¼ hepatocytes containing lipid droplets (1 point); there were not more than ½ hepatocytes containing lipid droplets (2 points); there were not more than ¾ hepatocytes containing lipid droplets (3 points); hepatic tissue was almost replaced by lipid droplets (4 points).

The test results are shown in Table 12.

TABLE 12
Effect of the composition of a glutathione precursor on liver
histopathology of mice (x̅ ± s)

| | Number of | |
|---|---|---|
| Group | animals (mice) | x̅ ± s |
| Blank control | 13 | 0.62 ± 0.77^{d} |
| Model control | 13 | 2.48 ± 0.86^{a} |
| Experimental group 1 | 13 | 1.46 ± 1.05^{bc} |
| Experimental group 2 | 13 | 1.90 ± 0.87^{ab} |
| Experimental group 3 | 13 | 1.92 ± 0.76^{ab} |
| Experimental group 4 | 13 | 1.61 ± 0.65^{bc} |
| Experimental group 5 | 13 | 1.49 ± 0.66^{bc} |
| Experimental group 6 | 13 | 1.15 ± 0.90^{cd} |
| Experimental group 7 | 13 | 1.32 ± 0.23^{bc} |

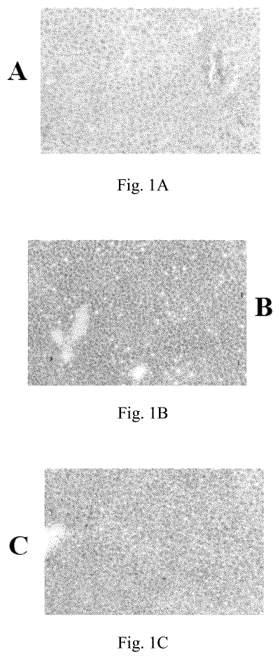

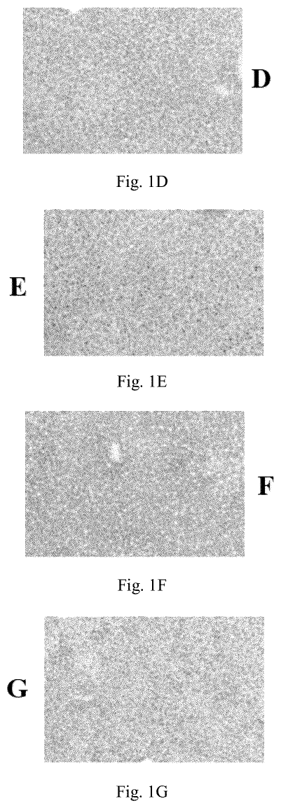

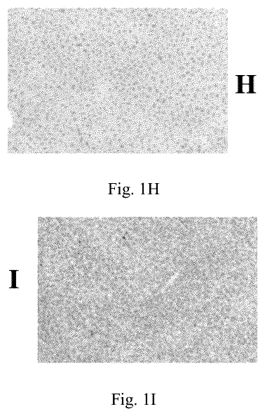

As can be seen from the experimental results, compared with the blank control group, the degeneration score of the liver fat cells had a statistically significant difference (P<0.01) in the model control group, and the animal test model was established, thereby successfully reproducing the alcoholic liver damage model of the experimental mice. Compared with the model group, the fatty degeneration degree of the hepatocytes decreased in the experimental group 1 and the experimental groups 4-7, with a statistically significant difference (P<0.05). The histopathological examination pictures of each group are shown in FIG. 1 (wherein, in FIG. 1, A-I correspond to A: livers in the blank control group (oil red O staining×200), B: livers in the model control group (oil red O staining×200), C: livers in the experiment group 1 (oil red O staining×200), D: livers in the experiment group 2 (oil red O staining×200), E: livers in the experiment group 3 (oil red O staining×200), F: livers in the experiment group 4 (oil red O staining×200), G: livers in the experiment group 5 (oil red O staining×200), H: livers in the experiment group 6 (oil red O staining×200), and I: livers in the experiment group 7 (oil red O staining×200)).

In conclusion, according to the determination principle of the Technical Specification for Inspection and Evaluation of Health Food (2003 edition) for the auxiliary protection effect on alcoholic liver damage, the experimental group 1 and the experimental groups 4-7 had an auxiliary protection effect on alcoholic liver damage.

### 2. Experiment for the Oxidation Resistance Function of the Composition of a Glutathione Precursor

In the experiment, the oxidation resistance effect of the composition was researched by detecting four indicators of MDA, protein carbonyl, antioxidant enzyme activity (GSH-PX or SOD) and reduced GSH content. If three of the four indicators were positive, the composition could be determined to have the oxidation resistance effect.

Test animals were as follows: male SPF-grade Kunming mice aged over 10 months, with body weights of 40-60 g, available from Hubei Provincial Laboratory Animal Public Service Center, Hubei, China [certification No.: SCXK (E) 2020-0018].

The experimental method was as follows: 104 mice were randomly divided into 8 groups, namely, a negative control group and experimental groups 1-7 (i.e., the formula of the test samples adopted the formula of the above experimental groups 1-7), with 13 mice in each group.

Distilled water was given to the blank control group, corresponding test substances (the dose of acetylcysteine or cysteine agent in each test substance was 500 mg/kg BW) were given to each experimental group, and the intragastric capacity was 20 mL/kg BW. After 30 days of continuous intragastric administration, blood was sampled from the inner canthus of each group of animals, a hemolysate was prepared, and the MDA content and the glutathione peroxidase (GSH-PX) activity were detected; the animals were sacrificed and the livers were taken as homogenates, 10% liver homogenates were prepared for reduced GSH and protein carbonyl determination, and 1% liver homogenates were prepared for superoxide dismutase (SOD) activity determination. Each detection kit was available from NanJing JianCheng Bioengineering Institute. The test results are shown in Table 13.

TABLE 13
Effect of the composition of a glutathione precursor on MDA, protein carbonyl,
SOD, GSH-Px and GSH (x̅ ± s, n = 13)

| | MDA | Protein carbonyl | GSH-PX | SOD | GSH |
|---|---|---|---|---|---|
| Group | (nmol/ml) | (nmol/mgprot) | (U/ml) | (U/mgprot) | (mg/gprot) |
| Negative control | 22.09 ± 3.80^{a} | 1.46 ± 0.53^{a} | 583.1 ± 28.9^{a} | 351.7 ± 38.8^{bc} | 24.86 ± 5.67^{b} |
| Experimental group 1 | 17.88 ± 2.55^{bc} | 1.33 ± 0.35^{ab} | 585.2 ± 24.2^{a} | 338.4 ± 44.9^{bc} | 30.05 ± 6.08^{a} |
| Experimental group 2 | 17.63 ± 4.52^{bc} | 1.27 ± 0.30^{abc} | 579.1 ± 19.0^{a} | 377.7 ± 53.2^{abc} | 28.41 ± 6.36^{ab} |
| Experimental group 3 | 19.01 ± 2.51^{b} | 1.04 ± 0.22^{cd} | 599.4 ± 8.7^{a} | 335.5 ± 47.0^{c} | 29.11 ± 5.47^{ab} |
| Experimental group 4 | 19.39 ± 2.51^{b} | 1.18 ± 0.27^{bc} | 581.6 ± 29.7^{a} | 338.1 ± 46.0^{bc} | 30.27 ± 3.90^{a} |
| Experimental group 5 | 17.12 ± 4.06^{bc} | 1.02 ± 0.19^{cd} | 581.3 ± 28.5^{a} | 374.9 ± 53.1^{abc} | 30.28 ± 3.85^{a} |
| Experimental group 6 | 15.46 ± 3.24^{c} | 0.89 ± 0.19^{d} | 583.9 ± 19.7^{a} | 394.1 ± 49.9^{a} | 31.91 ± 6.09^{a} |
| Experimental group 7 | 18.25 ± 2.97^{bc} | 1.02 ± 0.26^{cd} | 580.3 ± 18.7^{a} | 379.4 ± 52.1^{ab} | 30.62 ± 4.49^{a} |

As can be seen from the experimental results, the MDA content in 2% hemolysates of the aged mice significantly decreased in all experimental groups (P<0.05); the protein carbonyl content in 10% liver homogenates significantly decreased in the experimental groups 3-7 (p<0.05); the SOD activity in 1% liver homogenates of the aged mice significantly increased in the experimental group 6 (p<0.05); the reduced GSH content in 10% liver homogenates significantly increased in the experimental groups 1 and 4-7 (p<0.05). The experimental groups 4-7 had an oxidation resistance effect according to the determination principle.

### 3. Experiment for the Immunity Enhancement Function of the Composition of a Glutathione Precursor

In the experiment, the effect of the composition on the immune system of mice was researched from four aspects of cellular immune function, humoral immune function, monocyte-macrophage function and NK cell activity. If the results of any two of the four aspects were positive, the composition had the immunity enhancement effect.

Test animals were as follows: male SPF-grade Kunming mice aged 4 weeks, with body weights of 18-22 g, available from Hubei Provincial Laboratory Animal Public Service Center, Hubei, China [certification No.: SCXK (E) 2020-0018].

The experimental method was as follows: 400 mice were randomly divided into 5 groups, with 80 mice in each group, and each group was subjected to 5 kinds of immunoassays including body weight and organ/body ratio measurement, cellular immune function measurement, humoral immune function measurement, monocyte-macrophage function measurement, and NK cell activity measurement. For each immunoassay, 80 mice were tested and divided into 8 groups, namely a negative control group and experimental groups 1-7, with 10 mice in each group; distilled water was given to the negative control group, corresponding test substances (the dose of acetylcysteine or cysteine agent in each test substance was 500 mg/kg BW) were given to each experimental group, and the intragastric capacity was 20 mL/kg BW.

The body weights of the mice were weighed 1 h after the last administration, and then the following immune indicators were respectively measured according to the immune function detection program in the Technical Specification for Inspection and Evaluation of Health Food (2003 edition): ConA-induced splenic lymphocyte proliferation and transformation function, delayed allergy, number of antibody-producing cells, serum hemolysin level, ability of mouse peritoneal macrophage to phagocytize chicken erythrocyte, carbon clearance ability, and NK cell activity of mice.

### 3.1. Measurement of Humoral Immune Function

TABLE 14
Detection of the number of antibody-producing cells and the hemolysin
level of mice (x̅ ± s, n = 10)

| | Number of | |
|---|---|---|
| | hemolytic plaques | |
| | (N)/number of | |
| Group | whole splenocytes | Antibody product (mg) |
| Negative control | 149.50 ± 9.93^{d} | 91.23 ± 8.74^{d} |
| Experimental group 1 | 161.71 ± 11.52^{c} | 99.33 ± 4.61^{c} |
| Experimental group 2 | 155.15 ± 9.39^{cd} | 94.92 ± 5.00^{cd} |
| Experimental group 3 | 153.05 ± 5.68^{cd} | 95.86 ± 7.32^{cd} |
| Experimental group 4 | 160.61 ± 9.53^{c} | 98.09 ± 5.69^{c} |
| Experimental group 5 | 177.61 ± 8.28^{b} | 110.74 ± 6.31^{b} |
| Experimental group 6 | 228.73 ± 8.61^{a} | 165.32 ± 4.33^{a} |
| Experimental group 7 | 162.28 ± 11.55^{c} | 107.34 ± 5.32^{b} |

The results show that the number of hemolytic plaques and the antibody product in each experimental group were higher than those of the control group, the experimental groups 1 and 4-7 had significant differences (P<0.05) compared with the control group, and the number of hemolytic plaques and the antibody product in the experimental groups 5 and 6 were higher than those of the experimental group 1, with significant differences (P<0.05).

### 3.2. Monocyte-Macrophage Function

TABLE 15
Experiments for phagocytosis of chicken erythrocytes
by mouse peritoneal macrophages and carbon clearance
(x̅ ± s, n = 10)

| | | | Experiment |
|---|---|---|---|
| | Experiment for phagocytosis | | for carbon |
| | of chicken erythrocytes | | clearance |
| | Phagocytic | Phagocytic | Phagocytic |
| Group | index | rate (%) | index |
| Negative control | 1.25 ± 0.04^{d} | 31.35 ± 2.39^{c} | 4.09 ± 0.43^{c} |
| Experimental group 1 | 1.44 ± 0.08^{bc} | 33.88 ± 2.48^{b} | 4.63 ± 0.48^{b} |
| Experimental group 2 | 1.30 ± 0.03^{cd} | 32.37 ± 1.81^{bc} | 4.26 ± 0.23^{bc} |
| Experimental group 3 | 1.30 ± 0.03^{cd} | 34.36 ± 1.51^{b} | 4.34 ± 0.36^{bc} |
| Experimental group 4 | 1.50 ± 0.13^{ab} | 34.74 ± 3.11^{b} | 4.59 ± 0.42^{b} |
| Experimental group 5 | 1.51 ± 0.40^{ab} | 33.68 ± 1.46^{b} | 5.39 ± 0.42^{a} |
| Experimental group 6 | 1.65 ± 0.19^{a} | 40.62 ± 3.26^{a} | 5.35 ± 0.16^{a} |
| Experimental group 7 | 1.43 ± 0.09^{bc} | 33.97 ± 2.64^{b} | 4.56 ± 0.37^{b} |

As can be seen from Table 15, the phagocytic index and the phagocytic rate of the macrophages of the mice in each experimental group were higher than those of the control group, and there were significant differences between the groups through ANOVA; the experimental groups 1 and 4-7 had significant differences (P<0.05) compared with the control group, and the experimental group 6 had a significant difference compared with the experimental group 1 (P<0.05).

According to the experimental data of cellular immunity (ConA-induced splenic lymphocyte proliferation and transformation function and delayed allergy of mice) and NK cell activity, each experimental group had no significant difference compared with the negative control group through statistical analysis.

In conclusion, according to the determination principle of the Technical Specification for Inspection and Evaluation of Health Food (2003 edition) for the immunity enhancement effect, if the results of any two of the four aspects of cellular immune function, humoral immune function, monocyte-macrophage function and NK cell activity were positive, the formula compositions of the experimental groups 1 and 4-7 had the immunity enhancement effect.

Based on the above function experiments, the experimental groups 4-7 all could achieve the effects of liver protection, oxidation resistance and immunity enhancement, wherein the experimental group 6 achieved significant effects on liver protection (MDA reduction, increase of reduced GSH content, decrease in TG content, and liver histopathology), oxidation resistance (MDA reduction, protein carbonyl reduction, and SOD and GSH improvement), and immunity enhancement (humoral immunity function and mononuclear-macrophage function), and based on all of the indicators, the experimental group 6 achieved the best effect.

Although specific embodiments of the present disclosure have been described above, it should be understood by those skilled in the art that these embodiments are merely illustrative and that the protection scope of the present disclosure is defined by the appended claims. Various changes or modifications may be made to these embodiments by those skilled in the art without departing from the principle and spirit of the present disclosure, and such changes and modifications shall fall within the protection scope of the present disclosure.

1. A composition of a glutathione precursor, wherein the composition comprises the following components: a selenium-containing compound, a glutathione precursor, and a zinc-containing compound, wherein the composition of a glutathione precursor comprises the following components in parts by weight: 5-50 parts of the selenium-containing compound, 200-1200 parts of the glutathione precursor, and 20-105 parts of the zinc-containing compound.

2. The composition of a glutathione precursor according to claim 1, wherein the selenium-containing compound comprises one or more of sodium selenite, L-selenium-methylselenocysteine and a selenium-enriched yeast;
or the particle size of the selenium-containing compound is 40-120 meshes;
or the glutathione precursor comprises cysteine and/or N-acetylcysteine;
or the particle size of the glutathione precursor is 10-140 meshes;
or the zinc-containing compound comprises one or more of zinc gluconate, zinc sulfate, zinc citrate, zinc oxide and zinc lactate;
or the particle size of the zinc-containing compound is 40-120 meshes.

3. The composition of a glutathione precursor according to claim 1,
wherein the amount of the selenium-containing compound is 20-50 parts;
or, the amount of the glutathione precursor is 400-1200 parts by weight;
or, the amount of the zinc-containing compound is 30-105 parts by weight.

4. The composition of a glutathione precursor according to claim 1, wherein the composition of a glutathione precursor is selected from any one of i to x:
i. the composition of a glutathione precursor comprises 500-700 parts of N-acetylcysteine or cysteine, 5-30 parts of a selenium-enriched yeast or L-selenium-methylselenocysteine, and 20-70 parts of zinc lactate or zinc gluconate;
ii. the composition of a glutathione precursor comprises 25-35 parts of a selenium-enriched yeast, 500-700 parts of N-acetylcysteine, and 60-80 parts of zinc gluconate;
iii. the composition of a glutathione precursor comprises 30 parts of a selenium-enriched yeast, 600 parts of N-acetylcysteine, and 70 parts of zinc gluconate;
iv. the composition of a glutathione precursor comprises 45 parts of a selenium-enriched yeast, 600 parts of N-acetylcysteine, and 50 parts of zinc gluconate;
v. the composition of a glutathione precursor comprises 50 parts of a selenium-enriched yeast, 495 parts of N-acetylcysteine, and 105 parts of zinc lactate;
vi. the composition of a glutathione precursor comprises 30 parts of sodium selenite, 525 parts of cysteine, and 105 parts of zinc oxide;
vii. the composition of a glutathione precursor comprises 50 parts of sodium selenite, 515 parts of cysteine, and 105 parts of zinc sulfate;
viii. the composition of a glutathione precursor comprises 50 parts of sodium selenite, 450 parts of acetylcysteine, and 105 parts of zinc sulfate;
ix. the composition of a glutathione precursor comprises 5 parts of L-selenium-methylselenocysteine, 650 parts of cysteine, and 50 parts of zinc gluconate;
or x. the composition of a glutathione precursor comprises 5 parts of L-selenium-methylselenocysteine, 700 parts of N-acetylcysteine, and 20 parts of zinc lactate.

5. The composition of a glutathione precursor according to claim 1, wherein the composition of a glutathione precursor further comprises a component A comprising one or more of vitamins, taurine, fatty acids, proteins, probiotics, polypeptides, lipoic acid, coenzyme Q10, arginine, ornithine, citrulline, glutamine, isoleucine, valine, leucine, and histidine;
or the composition of a glutathione precursor further comprises a flavoring agent.

6. (canceled)

7. (canceled)

8. A pharmaceutical formulation, wherein raw materials thereof comprise the composition of a glutathione precursor according to claim 1.

9. The pharmaceutical formulation according to claim 8, wherein the raw materials of the pharmaceutical formulation further comprise one or more of a diluent, a binder, a disintegrant, a lubricant, a glidant, and a filler.

10. The pharmaceutical formulation according to claim 8, wherein the pharmaceutical formulation is a powder, a granule, a tablet, a capsule formulation, an oral liquid, a dry suspension, or other pharmaceutical formulations.

11. (canceled)

12. A method for protecting a liver, resisting oxidation or enhancing immunity, comprising administering to a patient a medicament comprising the composition of a glutathione precursor according to claim 1.

13. The composition of a glutathione precursor according to claim 2, wherein the particle size of the selenium-containing compound is 60-80 meshes;
or the particle size of the glutathione precursor is 20-80 meshes;
or the particle size of the zinc-containing compound is 60-80 meshes.

14. The composition of a glutathione precursor according to claim 1, wherein the composition of a glutathione precursor comprises a selenium-enriched yeast, N-acetylcysteine and zinc gluconate;
or, the composition of a glutathione precursor comprises a selenium-enriched yeast, N-acetylcysteine and zinc lactate;
or, the composition of a glutathione precursor comprises sodium selenite, cysteine and zinc oxide;
or, the composition of a glutathione precursor comprises sodium selenite, cysteine and zinc sulfate;
or, the composition of a glutathione precursor comprises L-selenium-methylselenocysteine, N-acetylcysteine and zinc lactate;
or, the composition of a glutathione precursor comprises L-selenium-methylselenocysteine, cysteine and zinc gluconate.

15. The composition of a glutathione precursor according to claim 1, wherein the amount of the selenium-containing compound is 30-50 parts;
or, the amount of the glutathione precursor is 600-800 parts by weight;
or, the amount of the zinc-containing compound is 70-105 parts by weight.

16. The composition of a glutathione precursor according to claim 5, wherein the particle size of the flavoring agent is 40-120 meshes;
or, when the composition of a glutathione precursor comprises the following components in parts by weight: 5-50 parts of the selenium-containing compound, 200-1200 parts of the glutathione precursor, and 20-105 parts of the zinc-containing compound, the content of the component A is 1-60 parts;
or, when the composition of a glutathione precursor comprises the following components in parts by weight: 5-50 parts of the selenium-containing compound, 200-1200 parts of the glutathione precursor, and 20-105 parts of the zinc-containing compound, the content of the flavoring agent is 10-30 parts;
or, the flavoring agent comprises one or more of a green tea essence, a sweet orange essence, and a blueberry essence.

17. The composition of a glutathione precursor according to claim 16, wherein when the flavoring agent is a green tea essence, the content of the green tea essence is 10-20 parts by weight;
or, when the flavoring agent is a sweet orange essence, the content of the sweet orange essence is 10-30 parts by weight;
or, when the flavoring agent is a blueberry essence, the content of the blueberry essence is 10-30 parts by weight.

18. The pharmaceutical formulation according to claim 9, wherein when the pharmaceutical formulation comprises the lubricant, the content of the lubricant is 3-8 parts by weight;
or when the pharmaceutical formulation comprises the glidant, the content of the glidant is 10-20 parts by weight;
or when the pharmaceutical formulation comprises the filler, the content of the filler is 1-100 parts by weight, or 1.25-37.5 parts by weigh, or 2.5-22.5 parts by weight;
or the particle size of the filler is 40-120 meshes or 60-80 meshes;
or wherein when the pharmaceutical formulation comprises the diluent, the content of the diluent is 1-40 parts by weight;
or wherein when the pharmaceutical formulation comprises the binder, the content of the binder is 1-30 parts by weight;
or wherein the diluent is sodium carboxymethyl starch;
or wherein the binder is povidone;
or wherein the filler is microcrystalline cellulose;
or wherein the lubricant is magnesium stearate;
or wherein the glidant is silicon dioxide and/or talcum powder.

19. The pharmaceutical formulation according to claim 10, wherein the pharmaceutical formulation is a capsule formulation, and raw materials of the capsule formulation further comprise a lubricant, a glidant, a flavoring agent, and a filler;
or the pharmaceutical formulation is a tablet, and raw materials of the tablet further comprise a lubricant, a glidant, a flavoring agent, and a filler.

20. The pharmaceutical formulation according to claim 19, wherein the capsule formulation is prepared by the following preparation method: filling a mixed powder of the glutathione precursor, the zinc-containing compound, the selenium-containing compound, the flavoring agent, the lubricant, the glidant and the filler into a capsule;
or, the capsule formulation is prepared by the following preparation method: mixing a first mixed powder of the zinc-containing compound, the selenium-containing compound, the flavoring agent and the filler with a mixed powder of the glutathione precursor to obtain a second mixed powder, then mixing the second mixed powder with the lubricant and the glidant to obtain a total mixed powder, and filling the total mixed powder into a capsule;
or, the tablet is prepared by the following preparation method: tabletting a mixed powder of the glutathione precursor, the zinc-containing compound, the selenium-containing compound, the flavoring agent, the lubricant, the glidant and the filler;
or, the tablet is prepared by the following preparation method: mixing a first mixed powder of the zinc-containing compound, the selenium-containing compound, the flavoring agent and the filler with a mixed powder of the glutathione precursor to obtain a second mixed powder, then mixing the second mixed powder with the lubricant and the glidant to obtain a total mixed powder, and tabletting the total mixed powder.

21. The pharmaceutical formulation according to claim 20, wherein the content of the lubricant is 3-8 parts by weight;
or wherein the content of the glidant is 10-20 parts by weight;
or wherein the content of the filler is 1-100 parts by weight, or 1.25-37.5 parts by weight, or 2.5-22.5 parts by weight, or 2.5-30.5 parts by weight;
or the particle size of the filler is 40-120 meshes or 60-80 meshes;
or before the first mixed powder is obtained, the zinc-containing compound and the selenium-containing compound are sieved through a 60-80-mesh sieve;
or before the first mixed powder is obtained, the filler is sieved through a 60-80-mesh sieve;
or before the first mixed powder is obtained, the flavoring agent is sieved through a 40-60-mesh sieve;
or before the second mixed powder is obtained, the glutathione precursor is sieved through a 20-80-mesh sieve or 40-80-mesh sieve;
or wherein the tablet can have a net weight of 0.75 g/tablet;
or wherein the tablet can have the hardness of 100-160 N.

22. The pharmaceutical formulation according to claim 20, wherein the capsule formulation is prepared by the following preparation method: filling a mixed powder of N-acetylcysteine, zinc gluconate, selenium-enriched yeast, green tea essence, magnesium stearate, silicon dioxide and microcrystalline cellulose into a capsule;
or, the capsule formulation is prepared by the following preparation method: mixing a first mixed powder of zinc gluconate, selenium-enriched yeast, green tea essence and microcrystalline cellulose with a mixed powder of N-acetylcysteine to obtain a second mixed powder, then mixing the second mixed powder with magnesium stearate and silicon dioxide to obtain a total mixed powder, and filling the total mixed powder into a capsule.

23. A method for protecting a liver, resisting oxidation or enhancing immunity, comprising administering to a patient a medicament comprising the pharmaceutical formulation according to claim 8.
